(19) United States
(12) Patent Application Publication
(10) Pub. No.: US 2024/0331870 A1
(43) Pub. Date: Oct. 3, 2024

(54) BMD MODEL TRAINING METHOD, BMD ABNORMALITY RISK PREDICTION METHOD, BMD ABNORMALITY RISK LEARNING SYSTEM, AND BMD ABNORMALITY RISK PREDICTION SYSTEM

(71) Applicant: Acer Medical Inc., NEW TAIPEI CITY (TW)

(72) Inventor: Chin-Han TSAI, NEW TAIPEI CITY (TW)

(21) Appl. No.: 18/610,398

(22) Filed: Mar. 20, 2024

(30) Foreign Application Priority Data
Mar. 29, 2023 (TW) 112111914

(51) Int. Cl. G16H 50/30; G16H 10/60

(52) U.S. Cl. CPC G16H 50/30; G16H 10/60

(57) ABSTRACT

A method of training a bone mineral density (BMD) model, including a training data retrieval step, a positioning step, and a model training step. The training data retrieval step retrieves one or more chest X-ray images with the BMD of a person. The positioning step locates and retrieves specific bone positions in the chest X-ray image. The model training step trains the AI model based on the bone positions in the chest X-ray image and the BMD. The specific bone position includes the first segment of the lumbar vertebrae. The BMD is the lumbar BMD value measured by dual-energy X-ray absorptiometry (DXA).

### CROSS REFERENCE TO RELATED APPLICATIONS

This Application claims priority of Taiwan Patent Application No. 112111914, filed on Mar. 29, 2023, the entirety of which is incorporated by reference herein.

### BACKGROUND OF THE INVENTION

### Field of the Invention

The present invention relates to a bone mineral density (BMD) model training method, a BMD abnormality risk prediction method, a BMD abnormality risk learning system, and a BMD abnormality risk prediction system.

### Description of the Related Art

The traditional methods used in BMD measurement require the use of dual-energy X-ray absorptiometry (DXA). Inventions that use dual-energy X-ray absorptiometry for training and predicting BMD are well known in the prior art. However, dual-energy X-ray absorptiometry is expensive, so it is not as widespread as chest X-ray machines in terms of equipment quantity. Moreover, dual-energy X-ray absorptiometry is bulky and cannot be set up in an X-ray machine van the way that chest X-ray machines can, to provide measurement for people during general health examinations.

To sum up, since dual-energy X-ray absorptiometry is not as popular as chest X-ray machines, the amount of training data that the dual-energy X-ray absorptiometry can obtain is far less than that of chest X-ray machines, making it an unfavorable option for improving the accuracy of the AI algorithm.

Furthermore, as the society continues to age, the number of people suffering from osteoporosis continues to increase. Osteoporosis does not cause obvious symptoms. However, when the bones are fragile and the risk of fracture is increased, minor injuries may cause fractures, as well as other illnesses and dysfunctions, and may even lead to death. Therefore, a more popular and accurate BMD measurement method is needed.

### BRIEF SUMMARY OF THE INVENTION

The present invention discloses a bone mineral density (BMD) model training method executed by a processing unit. The bone mineral density (BMD) model training method comprises a training data retrieval step, a positioning step, and a training step. The training data retrieval step obtains several sets of chest X-ray images and BMD values of the same person for use as training data. The positioning step locates and retrieves X-ray images of specific bone positions from the chest X-ray images. The training step trains a BMD AI model based on the X-ray images of the specific bone positions and the BMD values. Wherein the specific bone positions comprise a 1^{st} segment of lumbar vertebrae, and the BMD values are lumbar vertebra BMD values measured by dual-energy X-ray absorptiometry (DXA).

According to the BMD model training method disclosed in the present invention, the BMD value with high accuracy can be used as the benchmark (ground truth) of the training data, and the BMD AI model can be trained through chest X-ray images. Since the chest X-ray images are easier to obtain, the present invention can increase the amount of training data and thereby improve the accuracy of the BMD AI model.

In the BMD model training method mentioned above, the specific bone positions comprise a 12^{th} thoracic vertebra.

The 1^{st} lumbar vertebra and the 12^{th} thoracic vertebra are the bones that bear the weight of the human body. When there are symptoms of osteoporosis, the bones that bear the weight of the human body are more susceptible to osteoporosis than other bone areas. Therefore, using the 1^{st} lumbar vertebra and/or the 12^{th} thoracic vertebra position in chest X-ray images to train the BMD AI model can further improve the accuracy of the BMD AI model.

Furthermore, the present invention discloses a BMD abnormality risk prediction method executed by a processing unit. The BMD abnormality risk prediction method comprises: an inference data retrieval step, obtaining a chest X-ray image as inference data; a positioning step, locating and retrieving X-ray images of specific bone positions from the chest X-ray image; and an inference step, utilizing the BMD AI model and the X-ray images of the specific bone positions to generate a BMD prediction result, and output the BMD prediction result.

According to the BMD abnormality risk prediction method disclosed in the present invention, BMD can be predicted through a highly accurate BMD AI model.

The BMD abnormality risk prediction method mentioned above further comprises an evaluation step evaluating risk of BMD abnormality based on the BMD prediction result.

By evaluating the risk of BMD abnormality based on the BMD prediction results, a more concise and clear evaluation result can be presented to a user.

In the BMD abnormality risk prediction method mentioned above, the specific bone positions comprise a 12^{th} thoracic vertebra.

The 1^{st} lumbar vertebra and the 12^{th} thoracic vertebra are the bones that bear the weight of the human body. When there are symptoms of osteoporosis, the bones that bear the weight of the human body are more susceptible to osteoporosis than other bone areas. Therefore, using the 1^{st} lumbar vertebra and/or the 12^{th} thoracic vertebra position in chest X-ray images to infer BMD can further improve the accuracy of the inference results.

In addition, the present invention also discloses a BMD abnormality risk learning system, comprising: a non-volatile memory, storing a BMD abnormality risk learning application; and a processing unit, executing a BMD abnormality risk learning application to implement: a training data input module, obtaining many sets of chest X-ray images and BMD values of the same person for use as training data; a positioning module, locating and retrieving X-ray images of specific bone positions from the chest X-ray images; and a AI training module, training a BMD AI model based on the X-ray images of the specific bone positions and the BMD values; wherein the specific bone positions comprise a 1^{st} segment of lumbar vertebrae; and the BMD values are lumbar vertebra BMD values measured by dual-energy X-ray absorptiometry (DXA).

As set forth, highly accurate BMD values can be used as ground truth of training data, and the BMD AI model can be trained using chest X-ray images according to the bone abnormality risk learning system disclosed in the present invention. Since chest X-ray images are easier to obtain, the amount of training data can be increased, thereby improving the accuracy of the BMD AI model.

Furthermore, the present invention also discloses a BMD abnormality risk prediction system, comprising: a non-volatile memory, storing a BMD abnormality risk learning application; and a processing unit, executing a BMD abnormality risk learning application to implement: an inference data input module, obtaining a chest X-ray image as inference data; a positioning module, locating and retrieving X-ray images of specific bone positions from the chest X-ray image; and an inference module, utilizing the BMD AI model and the X-ray images of the specific bone positions to generate a BMD prediction result, and output the BMD prediction result; wherein the specific bone positions comprise a 1^{st} segment of lumbar vertebrae; and the BMD values are lumbar vertebra BMD values measured by dual-energy X-ray absorptiometry (DXA).

As set forth, BMD can be predicted through a highly accurate BMD AI model according to the BMD abnormality risk prediction system disclosed in the present invention.

### BRIEF DESCRIPTION OF THE DRAWINGS

FIG. 1 is a block diagram of an exemplary hardware structure of a BMD abnormality risk learning system 1 according to Embodiment 1 of the present invention;

FIG. 2 is a block diagram of an exemplary hardware structure of a BMD abnormality risk prediction system 1 according to Embodiment 1 of the present invention;

FIG. 3 is a schematic diagram of locating and retrieving specific bone positions in chest X-ray images;

FIG. 4 is a block diagram of an exemplary hardware structure of a BMD abnormality risk prediction system 2A according to Embodiment 3 of the present invention;

FIG. 5 is a flow chart of a BMD model training method according to Embodiment 2 of the present invention;

FIG. 6 is a flow chart of a BMD abnormality risk prediction method according to Embodiment 2 of the present invention; and

FIG. 7 is a flow chart of a BMD abnormality risk prediction method according to Embodiment 3 of the present invention.

### DETAILED DESCRIPTION OF THE INVENTION

The following illustrates embodiments of the present invention with reference to additional drawings. The disclosed embodiments are only examples, and the scope of the present invention is not limited thereto.

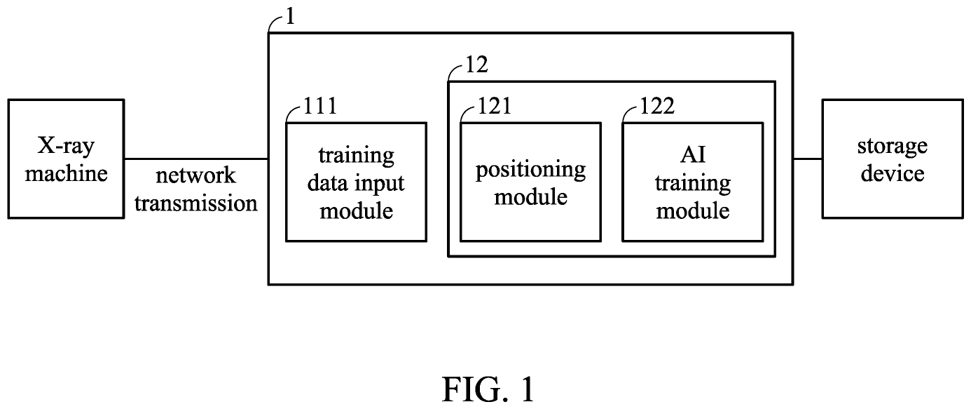

The following describes Embodiment 1 of the present invention with reference to FIG. 1. Embodiment 1 of the present invention is a bone mineral density (BMD) abnormality risk learning system 1. The BMD abnormality risk learning system 1 includes a training data input module 111, a positioning module 121, and an AI training module (BMD AI training module) 122.

The BMD abnormality risk learning system 1 can be implemented by a computing device (not shown) including at least one processing unit, memory, storage device and transmission interface.

For example, the processing unit is a hardware logic computing device such as central processing unit (CPU), digital signal processor (DSP), graphics processing unit (GPU), vision processing unit (VPU), application specific integrated circuit (ASIC) and/or field programmable gate array (FPGA), etc. The processing unit can also be a combination of the abovementioned logic computing devices.

The memory is a memory device that temporarily stores data. Specific examples are static random access memory (SRAM), dynamic random access memory (DRAM), or a register or cache memory of a processing unit, etc.

The storage device is a non-volatile memory device that stores data. A specific example is a solid state drive (SSD). The storage device may also be a hard disk drive (HDD), flash memory, etc. In Embodiment 1 of the present invention, the BMD abnormality risk learning application is stored in the storage device.

The transmission interface is a hardware interface that transmits data between processing units, memory, storage devices and other hardware. Specific examples are buses, signal lines, wired networks and/or wireless networks, etc. In Embodiment 1 of the present invention, the training data input module 111 can also input data through the transmission interface.

The BMD abnormality risk learning system 1 of Embodiment 1 of the present invention can be implemented by a processing unit executing software. It also can be implemented by dedicated hardware or firmware. Alternatively, part of it may be implemented by dedicated hardware or firmware, and the other part may be implemented by the processing unit executing software.

The following describes the training data input module 111, the positioning module 121 and the BMD AI training module 122 in the BMD abnormality risk learning system 1 in detail.

The training data input module 111 is a module for receiving training data. The training data includes multiple sets of chest X-ray images and BMD values of the same person. Specifically, the chest X-ray images and BMD value of the same person refer to the chest X-ray images of one person and the BMD of that person measured by a dual-energy X-ray absorptiometry. The above chest X-ray images can be poster-anterior view (PA view) X-ray images.

In Embodiment 1, the training data input module 111 receives the chest X-ray images of the same person from the X-ray machine through the network, and receives the BMD value of the same person through the network. The method of inputting the chest X-ray image and BMD value of the same person is not limited to this. For example, the training data input module 111 may also receive multiple sets of chest X-ray images and BMD values of the same person from a database. Alternatively, the training data input module 111 receives a person's chest X-ray image from the chest X-ray machine and the BMD value input by the user.

In a modification of Embodiment 1, in addition, the chest X-ray image can also be randomly rotated (e.g., 0 to 10 degrees) or randomly scaled (e.g., zoomed by 1% to 5%) to generate multiple chest X-ray images as training data. That is, a set of chest X-ray images and BMD values of the same person includes multiple chest X-ray images generated by rotating and/or scaling the chest X-ray images, as well as the BMD value of the person. In this way, the amount of training data can be increased and the accuracy of the trained AI model can be improved.

In another modification of Embodiment 1, there may be multiple chest X-ray images of the same person. That is, a set of chest X-ray images and BMD values of the same person includes a plurality of chest X-ray images taken by the person within a certain period of time, as well as the BMD values of the person measured during the certain period of time. For example, a set of chest X-ray images and BMD values of the same person may include multiple chest X-ray images of the same person within three months, as well as the BMD values of the person measured within the three months.

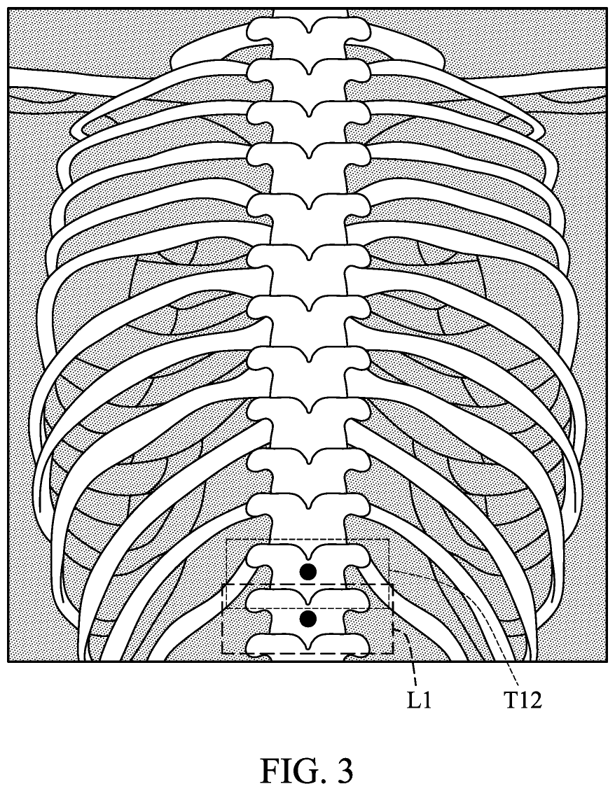

The following illustrates the positioning module 121 in the BMD abnormality risk learning system 1. The positioning module 121 is configured to locate specific bone positions in chest X-ray images. For example, FIG. 3 shows a schematic diagram of locating and retrieving the 12^{th} thoracic vertebra (T12) and the 1^{st} segment of the lumbar vertebrae (L1) in a chest X-ray image. The method of locating a specific bone position and the method of retrieving an image of a specific bone position can be achieved by, for example, Self-Cure-Network (SCN).

The positions of the 1^{st} segment of the lumbar vertebrae and the 12^{th} thoracic vertebra are the bone positions that bear the weight of the human body. The BMD of bones that bear the body's weight (e.g., the 1^{st} to 4^{th} lumbar vertebrae) is more susceptible to osteoporosis than other bone areas. Therefore, compared to bone areas such as the clavicle, cervical spine, or ribs, using the bone area that bears the weight of the human body to train the BMD AI model can improve the accuracy of the inference results. It is known that the correlation between the positions of the 1^{st} lumbar vertebra and the 12^{th} thoracic vertebra and the 1^{st} to 4^{th} lumbar vertebrae is as high as 0.9 or more. In a modification of Embodiment 1, X-ray images of the bone area of the 1^{st} lumbar vertebra and/or the 12^{th} thoracic vertebra can be located and retrieved as training data.

In addition, in another modification of Embodiment 1, the entire chest X-ray image can also be used for training the AI model. That is, the positioning module 121 retrieves the entire chest X-ray image as training data.

The following describes the AI training module 122 in the BMD abnormality risk learning system 1. The AI training module 122 trains the BMD AI model based on the X-ray image of the specific bone position and the BMD value retrieved by the positioning module 121. For example, the AI training module 122 may be densely connected convolutional network (DENSENET). For example, traditional regression algorithms such as support vector regression (SVR) and linear regression can be used for training the BMD AI model. In addition, deep learning can also be used for training the BMD AI model. The trained BMD AI model is output and stored in a storage device (non-volatile memory).

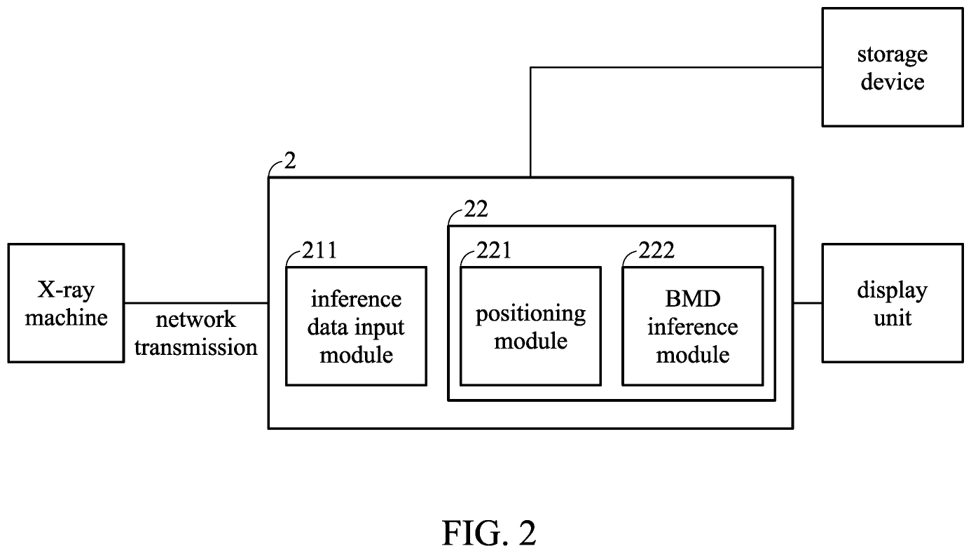

The following describes Embodiment 2 of the present invention with reference to FIG. 2. Embodiment 2 of the present invention is a BMD abnormality risk prediction system 2. The BMD abnormality risk prediction system 2 includes an inference data input module 211, a positioning module 221, and a BMD inference module 222.

The BMD abnormality risk prediction system 2 can be implemented by a computing device (not shown) including at least one processing unit, memory, storage device and transmission interface. The structure of the processing unit, memory, storage device and transmission interface can be as described in Embodiment 1, and the description will not be repeated here.

In Embodiment 2 of the present invention, the BMD abnormality risk prediction application is stored in a storage device (non-volatile memory). In addition, the inference data input module 211 can also input data through the transmission interface.

The BMD abnormality risk prediction system 2 in Embodiment 2 of the present invention can be implemented by a processing unit executing software, or can also be implemented by dedicated hardware or firmware. Alternatively, part of it may be implemented by dedicated hardware or firmware, and the other part may be implemented by the processing unit executing software.

The BMD abnormality risk prediction system 2 is a system that inputs the chest X-ray image of a BMD testing subject into, for example, the BMD AI model of Embodiment 1, and outputs the inferred BMD. The following describes the inference data input module 211, the positioning module 221 and the BMD inference module 222 in the BMD abnormality risk prediction system 2 in detail.

The inference data input module 211 obtains the chest X-ray image of the BMD testing subject as the input inference data. For example, the inference data input module 211 receives a chest X-ray image of the BMD testing subject from a chest X-ray machine or a database. The method of inputting inference data is not limited to this.

The type of chest X-ray image of the abovementioned subject is the same as the type of chest X-ray image used in the training of the BMD AI model in the BMD inference module 222. For example, if the PA view X-ray image is used when training the BMD AI model, then the inference data input here is also the PA view X-ray image of the BMD testing subject.

The following describes the positioning module 221 of Embodiment 2. The positioning module 221 locates and retrieves the X-ray image of a specific bone position in the chest X-ray image of the BMD testing subject. The positioning module 221 in Embodiment 2 may be the same as the positioning module 121 in Embodiment 1 and/or in the modifications of Embodiment 1. Therefore, the description will not be repeated here.

The BMD inference module 222 is configured to access the BMD AI model trained in Embodiment 1. The BMD inference module 222 inputs the BMD testing subject's chest X-ray image obtained by the inference data input module 211 into the BMD AI model, generates a BMD prediction result, and outputs the BMD prediction result. The BMD prediction result is a numerical value of BMD. The BMD prediction results can be displayed through a display unit.

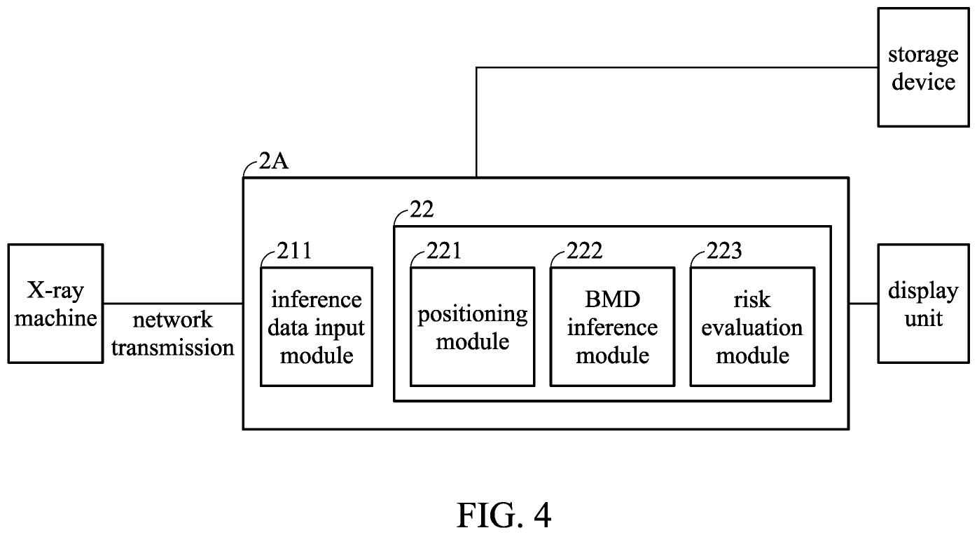

Embodiment 3 is a modification of Embodiment 2. The following illustrates a BMD abnormality risk prediction system 2A in Embodiment 3 of the present invention with reference to FIG. 4. The BMD abnormality risk prediction system 2A includes an inference data input module 211, a positioning module 221, a BMD inference module 222, and a risk evaluation module (evaluation module) 223. The following describes the differences between Embodiment 3 and Embodiment 2.

The BMD abnormality risk prediction system 2A further includes a risk evaluation module 223 evaluating the risk of BMD abnormality based on the BMD prediction results. For example, the risk evaluation module 223 converts the BMD value output by the BMD inference module 222 into a T-score and/or a Z-score. The T-score value is the number of standard deviations between the subject's BMD and the average BMD of young people of the same gender (about 20 to 29 years old). The Z-Score value is the number of standard deviations between the subject's BMD and the average BMD of people of the same gender and age. When T-scores≤−2.5 or Z-score≤−2.0, the chest X-ray image is evaluated to be a chest X-ray image with a high risk of BMD abnormality. The evaluation result of the risk evaluation module 223 can be displayed through a display unit.

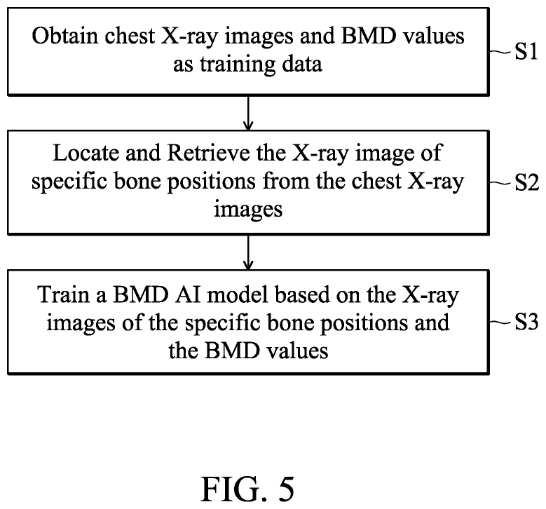

The following describes the BMD model training method in Embodiment 4 of the present invention with reference to FIG. 5. FIG. 5 is a flow chart of the BMD model training method of Embodiment 4.

In step S1, the processing unit obtains training data. The training data includes multiple sets of chest X-ray images and BMD values of the same person. The chest X-ray image and BMD value of the same person refer to the chest X-ray image of one person and the BMD of that person measured by a dual-energy X-ray absorptiometry. The above chest X-ray images can use PA view X-ray images.

For example, the processing unit may receive multiple sets of chest X-ray images and BMD values of the same person from a database. Alternatively, the processing unit may receive a chest X-ray image of a person from a chest X-ray machine, and receive a BMD value input by a user. The method of inputting the chest X-ray image and BMD value of the same person is not limited to this.

In a modification, the chest X-ray image can also be randomly rotated (e.g., rotated 0 to 10 degrees), or can be randomly scaled (e.g., zoomed by 1% to 5%) to generate multiple chest X-rays as training data. In this way, the amount of training data can be increased and the accuracy of the trained AI model can be improved.

In another modification, there may be multiple chest X-ray images of the same person. For example, a set of chest X-ray images and BMD values of the same person may include multiple chest X-ray images of the same person within three months, as well as the person's BMD value measured within the three months.

In step S2, the processing unit locates and retrieves specific bone positions in the above-mentioned chest X-ray image. The method of locating a specific bone position and the method of retrieving an image of a specific bone position can be achieved by, for example, SCN. In a modification, the entire chest X-ray image can also be used for training the AI model.

In step S3, the processing unit trains the BMD AI model based on the X-ray image of the specific bone position retrieved in step S2 and the BMD value obtained in step S1. The method of training the BMD AI model can use, for example, DENSENET. Traditional regression algorithms (e.g., SVR and linear regression) can also be used for training the BMD AI model. In addition, deep learning can also be used for training the BMD AI model. The trained BMD AI model is stored in a storage device (non-volatile memory).

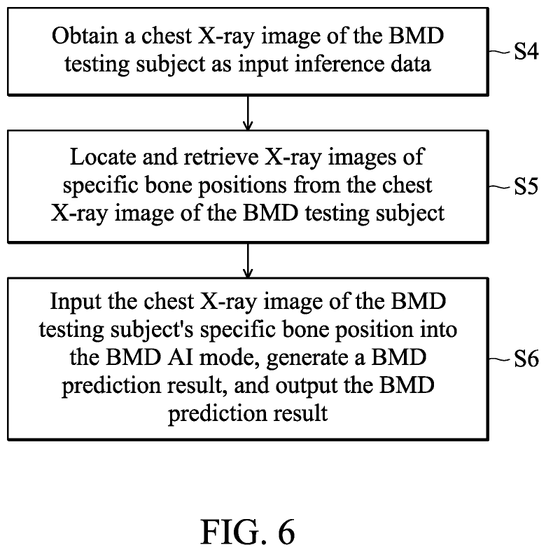

The following describes the BMD abnormality risk prediction method in Embodiment 5 of the present invention with reference to FIG. 6. FIG. 6 is a flow chart of the BMD abnormality risk prediction method in Embodiment 5.

In step S4, the processing unit obtains the chest X-ray image of the BMD testing subject as input inference data. For example, the processing unit receives the chest X-ray image of the BMD testing subject from a chest X-ray machine, or receives the chest X-ray image of the BMD testing subject from a database. The method of inputting inference data is not limited to this.

In step S5, the processing unit locates and retrieves the X-ray image of the specific bone position from the chest X-ray image of the abovementioned BMD testing subject. The method of locating and retrieving the X-ray image of a specific bone position can be as described in Embodiment 4 and/or various modifications of Embodiment 4. The description will not be repeated here.

In step S6, the processing unit inputs the chest X-ray image of the BMD testing subject's specific bone position obtained in step S4 into the BMD AI model trained in Embodiment 4, generates the BMD prediction result, and outputs the BMD prediction result. The BMD prediction result is a numerical value of BMD. The BMD prediction results can be displayed through a display unit.

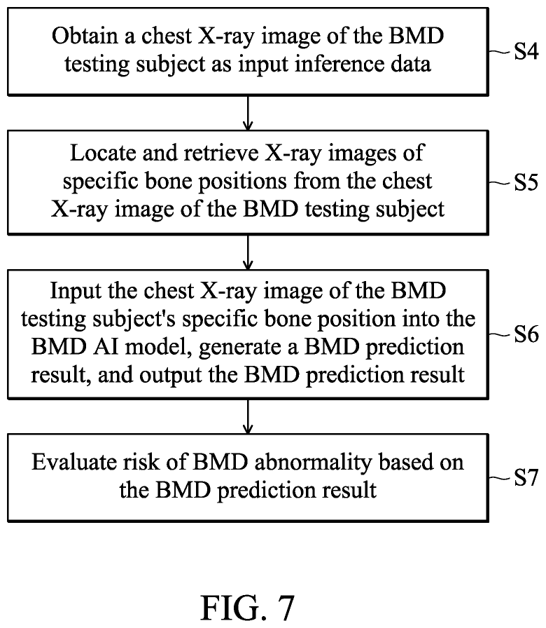

FIG. 7 is a flow chart of the BMD abnormality risk prediction method of Embodiment 3. Embodiment 6 is a modification of Embodiment 5. The following describes the differences between Embodiment 6 and Embodiment 5 with reference to FIG. 7.

Compared with Embodiment 5, Embodiment 6 adds step S7. In step S7, the processing unit evaluates the risk of BMD abnormality based on the BMD prediction result obtained in step S6. For example, the risk evaluation module 223 converts the BMD value output by the BMD inference module 222 into a T-score and/or a Z-score. When T-scores≤−2.5 or Z-scores≤−2.0, the chest X-ray image is evaluated to be a chest X-ray image with a high risk of BMD abnormality. The evaluation result of the risk evaluation module 223 can be displayed through a display unit.

Although a plurality of embodiments are described separately, the abovementioned embodiments may also be implemented in combination. Alternatively, one of them may be partially implemented in multiple embodiments. Alternatively, the plurality of embodiments may also be partially combined. In addition, the structures and steps of the plurality of embodiments described above may be partially modified as necessary.

Each embodiment described above is for making the present invention easy to understand. The above description is not intended to limit the present invention. Therefore, each element disclosed in each embodiment described above is intended to include all design changes or equivalents that fall within the technical scope of the present invention.

What is claimed is:

1. A bone mineral density (BMD) model training method executed by a processing unit, comprising:
a training data retrieval step, obtaining a plurality of sets of chest X-ray images and BMD values of the same person as training data;
a positioning step, locating and retrieving X-ray images of specific bone positions from the chest X-ray images; and
a training step, training a BMD AI model based on the X-ray images of the specific bone positions and the BMD values;
wherein the specific bone positions comprise a 1^{st} segment of lumbar vertebrae; and
the BMD values are lumbar vertebra BMD values measured by dual-energy X-ray absorptiometry (DXA).

2. The BMD model training method as claimed in claim 1, wherein the specific bone positions comprise a 12^{th} thoracic vertebra.

3. A BMD abnormality risk prediction method executed by a processing unit, comprising:
an inference data retrieval step, obtaining a chest X-ray image as inference data;
a positioning step, locating and retrieving X-ray images of specific bone positions from the chest X-ray image; and
an inference step, utilizing the BMD AI model as claimed in the BMD model training method of claim 1 and the X-ray images of the specific bone positions to generate a BMD prediction result, and output the BMD prediction result.

4. The BMD abnormality risk prediction method as claimed in claim 3, further executed by a processing unit, comprising:
an evaluation step, evaluating risk of BMD abnormality based on the BMD prediction result.

5. The BMD abnormality risk prediction method as claimed in claim 3, wherein the specific bone positions further comprise a 12^{th} thoracic vertebra.

6. A BMD abnormality risk learning system, comprising:
a non-volatile memory, storing a BMD abnormality risk learning application; and
a processing unit, executing a BMD abnormality risk learning application to implement:
a training data input module, obtaining a plurality of sets of chest X-ray images and BMD values of the same person as training data;
a positioning module, locating and retrieving X-ray images of specific bone positions from the chest X-ray images; and
a AI training module, training a BMD AI model based on the X-ray images of the specific bone positions and the BMD values;
wherein the specific bone positions comprise a 1^{st} segment of lumbar vertebrae; and
the BMD values are lumbar vertebra BMD values measured by dual-energy X-ray absorptiometry (DXA).

7. The BMD abnormality risk learning system as claimed in claim 6, wherein the specific bone positions further comprise a 12^{th} thoracic vertebra.

8. A BMD abnormality risk prediction system, comprising:
a non-volatile memory, storing a BMD abnormality risk learning application; and
a processing unit, executing a BMD abnormality risk learning application to implement:
an inference data input module, obtaining a chest X-ray image as inference data;
a positioning module, locating and retrieving X-ray images of specific bone positions from the chest X-ray image; and
an inference module, utilizing the BMD AI model and the X-ray images of the specific bone positions to generate a BMD prediction result, and output the BMD prediction result;
wherein the specific bone positions comprise a 1^{st} segment of lumbar vertebrae; and
the BMD values are lumbar vertebra BMD values measured by dual-energy X-ray absorptiometry (DXA).

9. The BMD abnormality risk prediction system as claimed in claim 8, wherein the processing unit further implements an evaluation module evaluating risk of BMD abnormality based on the BMD prediction results.

10. The BMD abnormality risk prediction system as claimed in claim 8, wherein the specific bone positions further comprise a 12^{th} thoracic vertebra.
